(19) United States
(12) Patent Application Publication
(10) Pub. No.: US 2022/0351483 A1
(43) Pub. Date: Nov. 3, 2022

(54) IMAGE PROCESSING SYSTEM, ENDOSCOPE SYSTEM, IMAGE PROCESSING METHOD, AND STORAGE MEDIUM

(71) Applicant: OLYMPUS CORPORATION, Tokyo (JP)

(72) Inventor: Fumiyuki SHIRATANI, Sagamihara-shi (JP)

(73) Assignee: OLYMPUS CORPORATION, Tokyo (JP)

(21) Appl. No.: 17/857,363

(22) Filed: Jul. 5, 2022

Related U.S. Application Data
(63) Continuation of application No. PCT/JP2020/000375, filed on Jan. 9, 2020.

(51) Int. Cl. G06V 10/25; G06V 10/764; G06V 10/774

(52) U.S. Cl. CPC G06V 10/25; G06V 10/764; G06V 10/774

(57) ABSTRACT

An image processing system includes a processor configured to acquire a processing target image, perform a classification process that classifies an observation method when the processing target image is captured to a first observation method or a second observation method based on an observation method classifier, perform a selection process that selects one of a plurality of detectors of region of interest for detecting a region of interest including a first detector of region of interest and a second detector of region of interest based on a classification result of the observation method classifier, and output a detection result based on the selected detector of region of interest.

### CROSS REFERENCE TO RELATED APPLICATION

This application is a continuation of International Patent Application No. PCT/JP2020/000375, having an international filing date of Jan. 9, 2020, which designated the United States, the entirety of which is incorporated herein by reference.

### BACKGROUND

A method of performing an image process targeting an in-vivo image to support a doctor's diagnosis has been widely known. Specifically, an attempt has been made to apply image recognition by deep learning to detection of a lesion and differentiation of a degree of malignancy. In addition, various kinds of methods for increasing accuracy of image recognition have also been disclosed.

For example, in Japanese Unexamined Patent Application Publication No. 2004-351100, comparison determination between a feature amount of a plurality of images that has already been classified to a normal image or an abnormal image and a feature amount of a newly input image is used for determination of a candidate for an abnormal shadow, whereby an attempt is made to increase accuracy of determination.

Japanese Unexamined Patent Application Publication No. 2004-351100 does not take into consideration of an observation method at the time of training and a detection process, and fails to disclose a method of changing a way of extracting a feature amount or a way of performing comparison determination in accordance with the observation method.

### SUMMARY

In accordance with one of some aspect, there is provided an image processing system comprising a processor including hardware, the processor being configured to acquire a processing target image, perform a classification process that classifies an observation method when the processing target image is captured to one of a plurality of observation methods including a first observation method and a second observation method based on an observation method classifier, perform a selection process that selects one of a plurality of detectors of region of interest including a first detector of region of interest and a second detector of region of interest based on a classification result of the observation method classifier, each detector of region of interest detecting a region of interest from the processing target image, output, when the first detector of region of interest is selected in the selection process, a detection result in which the region of interest is detected from the processing target image classified to the first observation method based on the first detector of region of interest, and output, when the second detector of region of interest is selected in the selection process, the detection result in which the region of interest is detected from the processing target image classified to the second observation method based on the second detector of region of interest.

In accordance with one of some aspect, there is provided an endoscope system comprising: an imaging device configured to capture an in-vivo image; and a processor including hardware, wherein the processor acquires the in-vivo image as a processing target image, performs a classification process that classifies an observation method when the processing target image is captured to one of a plurality of observation methods including a first observation method and a second observation method based on an observation method classifier, performs a selection process that selects one of a plurality of detectors of region of interest including a first detector of region of interest and a second detector of region of interest based on a classification result of the observation method classifier, each detector of region of interest detecting a region of interest from the processing target image, outputs, when the first detector of region of interest is selected in the selection process, a detection result in which the region of interest is detected from the processing target image classified to the first observation method based on the first detector of region of interest, and outputs, when the second detector of region of interest is selected in the selection process, the detection result in which the region of interest is detected from the processing target image classified to the second observation method based on the second detector of region of interest.

In accordance with one of some aspect, there is provided an image processing method, comprising: acquiring a processing target image, performing a classification process that classifies an observation method when the processing target image is captured to one of a plurality of observation methods including a first observation method and a second observation method based on an observation method classifier, performing a selection process that selects one of a plurality of detectors of region of interest including a first detector of region of interest and a second detector of region of interest based on a classification result of the observation method classifier, each detector of region of interest detecting a region of interest from the processing target image, outputting, when the first detector of region of interest is selected in the selection process, a detection result in which the region of interest is detected from the processing target image classified to the first observation method based on the first detector of region of interest, and outputting, when the second detector of region of interest is selected in the selection process, the detection result in which the region of interest is detected from the processing target image classified to the second observation method based on the second detector of region of interest.

According to another aspect of the invention, there is provided a computer readable non-transitory storage medium that stores a program that causes a computer to execute steps comprising: acquiring a processing target image, performing a classification process that classifies an observation method when the processing target image is captured to one of a plurality of observation methods including a first observation method and a second observation method based on an observation method classifier, performing a selection process that selects one of a plurality of detectors of region of interest including a first detector of region of interest and a second detector of region of interest based on a classification result of the observation method classifier, each detector of region of interest detecting a region of interest from the processing target image, outputting, when the first detector of region of interest is selected in the selection process, a detection result in which the region of interest is detected from the processing target image classified to the first observation method based on the first detector of region of interest, and outputting, when the second detector of region of interest is selected in the selection process, the detection result in which the region of interest is detected from the processing target image classified to the second observation method based on the second detector of region of interest.

### BRIEF DESCRIPTION OF THE DRAWINGS

FIG. 1 illustrates a schematic configuration example of a system including an image processing system.

FIG. 2 illustrates a configuration example of a training device.

FIG. 3 illustrates a configuration example of the image processing system.

FIG. 4 illustrates a configuration example of an endoscope system.

FIGS. 5A and 5B each illustrate a configuration example of a neural network.

FIG. 6A is a diagram for describing an input to and an output from a detector of region of interest. FIG. 6B is a diagram for describing an input to and output from an observation method classifier.

FIG. 7 illustrates a configuration example of a training device in accordance with a first embodiment.

FIG. 8 illustrates a configuration example of an image processing system in accordance with the first embodiment.

FIG. 9 is a flowchart describing a detection process in accordance with the first embodiment.

FIG. 10 illustrates a configuration example of a neural network, which is a detection-integrated-type observation method classifier.

FIG. 11 illustrates a configuration example of an image processing system in accordance with a second embodiment.

FIG. 12 is a flowchart describing a detection process in accordance with the second embodiment.

FIG. 13 illustrates a configuration example of a training device in accordance with a first embodiment.

FIG. 14 illustrates a configuration example of a training device in accordance with a first embodiment.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

1. Overview

When a doctor makes a diagnosis using an endoscope system, various kinds of observation methods are used. The observation mentioned herein is, specifically, to see a state of a subject using a captured image. The captured image is, specifically, an in-vivo image. The observation method changes depending on a type of illumination light of an endoscope apparatus and the state of the subject. As the observation method, normal light observation, special light observation, pigment spray observation, and the like can be assumed. In the normal light observation method, normal light is emitted as illumination light and image-capturing is thereby performed. In the special light observation method, special light is emitted as illumination light and image-capturing is thereby performed. In the pigment spray observation method, image-capturing is performed in a state where a dye is sprayed onto a subject. In the following description, an image captured in normal light observation is referred to as a normal light image, an image captured in special light observation is referred to as a special light image, and an image captured in pigment spray observation is referred to as a pigment-sprayed image.

The normal light is light having an intensity in a wide wavelength band out of wavelength bands corresponding to visible light, and is white light in a more limited sense. The special light is light having spectral characteristics different from those of the normal light, and is, for example, narrow band light having a wavelength band that is narrower than that of the normal light. Conceivable examples of an observation method using the special light include a narrow band imaging (NBI) method using narrow band light corresponding to a wavelength of 390 to 445 nm and narrow band light corresponding to a wavelength of 530 to 550 nm. The special light may include light having a wavelength band of light other than visible light such as infrared light. As the special light used for the special light observation, light having various kinds of wavelength bands has been known and a wide range of light is applicable to the present embodiment. A die used in the pigment spray observation is, for example, indigocarmine. Dispersing the indigocarmine can increase visibility of a polyp. Various kinds of die and various combinations of regions of interest to be targeted have been known, and a wide range of them is applicable to the pigment spray observation in accordance with the present embodiment.

As described above, for the purpose of supporting a doctor's diagnosis, an attempt has been made to create a detector by machine learning such as deep learning and apply the detector to detection of a region of interest. Note that the region of interest (regions of interest) in accordance with the present embodiment is a region in which the order of priority in imaging for a user is relatively higher than that in other regions. In a case where the user is a doctor who performs diagnosis or treatment, the region of interest corresponds to, for example, a region that shows a lesion portion. Note that if a target on which the doctor wants to perform imaging is bubbles or feces, the region of interest may be a region that shows a bubble portion or a feces portion. That is, while a target to which the user should pay attention is different depending on a purpose of imaging, on the occasion of the imaging, a region where the order of priority in imaging for the user is relatively higher than that in the other regions is the region of interest. The following description will be mainly given of an example in which the region of interest is a lesion or a polyp.

During endoscopic examination, an observation method for capturing an image of a subject is changed by the doctor's switching of illumination light between normal light and special light, spray of the pigment into body tissues, or the like. Due to the change in observation method, a detector's parameter appropriate for detection of the lesion is changed. For example, it is assumed that a detector that has been trained using only the normal light image produces an unfavorable result in terms of accuracy of detecting the lesion in the special light image as compared with the normal light image. For this reason, required is a method of desirably maintaining the accuracy of detection of the lesion even in a case where the observation method is changed during the endoscopic examination.

In a conventional method described in Japanese Unexamined Patent Application Publication No. 2004-351100 or the like, nothing is disclosed about what kind of image is used as training data to generate a detector, nor about, in a case where a plurality of detectors is generated, which combination of the plurality of detectors is made to execute a detection process.

A method in accordance with the present embodiment includes execution of a detection process of detecting the region of interest (regions of interest) based on a first detector of region of interest generated based on an image captured in a first observation method, and a second detector of region of interest generated based on an image captured in a second observation method. At this time, an observation method for a processing target image is estimated based on an observation method classification section, and a detector to be used for the detection process is selected based on a result of estimation. This enables execution of the detection process targeting the processing target image with high accuracy even in a case where the observation method for the processing target image is changed in various manners.

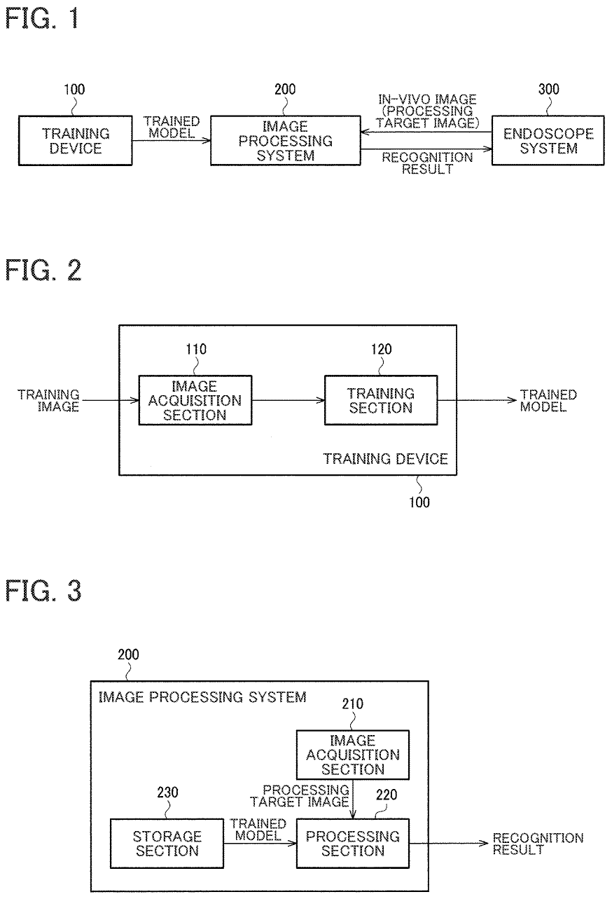

First, an outline configuration of a system including an image processing system 200 in accordance with the present embodiment will be described below with reference to FIGS. 1 to 4. Thereafter, a specific method and the flow of processing will be described in first to fourth embodiments.

FIG. 1 illustrates a configuration example of the system including the image processing system 200. The system includes a training device 100, an image processing system 200, and an endoscope system 300. Note that a configuration of the system is not limited to that illustrated in FIG. 1. Various modifications can be made such as omission of part of constituent elements and addition of another constituent element.

The training device 100 performs machine learning to generate a trained model. The endoscope system 300 causes an endoscope imaging device to capture an in-vivo image. The image processing system 200 acquires the in-vivo image as a processing target image. The image processing system 200 then operates in accordance with the trained model generated by the training device 100 to perform a detection process of detecting a region of interest (region of interests) targeting the processing target image. The endoscope system 300 acquires and displays a detection result. This enables implementation of the system that supports the doctor's diagnosis and the like using the machine learning.

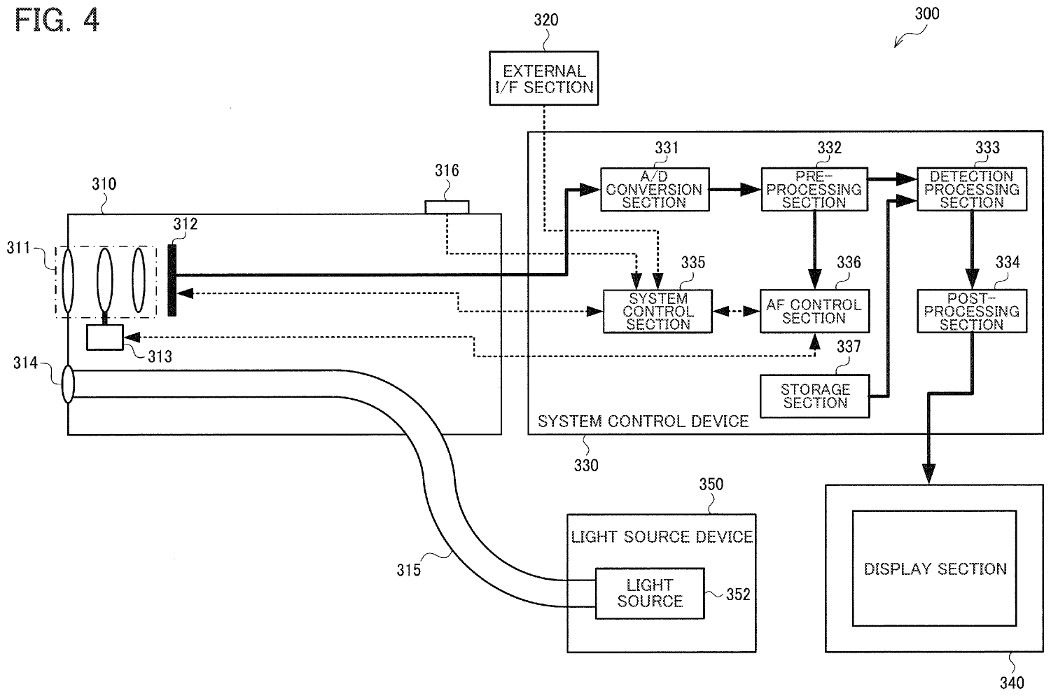

The training device 100, the image processing system 200, and the endoscope system 300, for example, may be arranged as individual devices. Each of the training device 100 and the image processing system 200 is, for example, an information processing device such as a personal computer (PC) and a server system. Note that the training device 100 may be implemented by a distributed process performed by a plurality of devices. For example, the training device 100 may be implemented by cloud computing using a plurality of servers. The image processing system 200 may be similarly implemented by cloud computing or the like. The endoscope system 300 is a device including an insertion section 310, a system control device 330, and a display section 340, as described later with reference to, for example, FIG. 4. Note that part or the whole of the system control device 330 may be implemented by equipment via a network of a server system or the like. For example, part or the whole of the system control device 330 is implemented by cloud computing.

In addition, one of the image processing system 200 and the training device 100 may include the other of the image processing system 200 and the training device 100. In this case, the image processing system (training device 100) is a system that performs machine learning to execute both the process of generating the trained model and the detection process in accordance with the trained model. Alternatively, one of the image processing system 200 and the endoscope system 300 may include the other of the image processing system 200 and the endoscope system 300. For example, the system control device 330 of the endoscope system 300 includes the image processing system 200. In this case, the system control device 330 executes both control of each section of the endoscope system 300 and the detection process in accordance with the trained model. Alternatively, a system including all of the training device 100, the image processing system 200, and the system control device 330 may be implemented. For example, a server system comprising one or more servers may perform the process of performing the machine learning to generate the trained model, the detection process in accordance with the trained model, and control of each section of the endoscope system 300. As described above, the specific configuration of the system illustrated in FIG. 1 can be modified in various manners.

FIG. 2 illustrates a configuration example of the training device 100. The training device 100 includes an image acquisition section 110 and a training section 120. The image acquisition section 110 acquires a training image. The image acquisition section 110 is, for example, a communication interface for acquiring the training image from another device. The training image is, for example, an image in which the normal light image, the special light image, or the pigment-sprayed image is provided with ground truth (correct data) as metadata. The training section 120 performs machine learning based on the acquired training image to generate the trained model. Details of data used for the machine learning and the specific flow of the training process will be described later.

The training section 120 comprises the following hardware. The hardware can include at least one of a digital signal processing circuit or an analog signal processing circuit. For example, the hardware can comprise one or more circuit devices mounted on a circuit board, or one or more circuit elements. The one or more circuit devices are, for example, integrated circuits (ICs), field-programmable gate array (FPGA) circuits, or the like. The one or more circuit elements are, for example, resistors, capacitors, or the like.

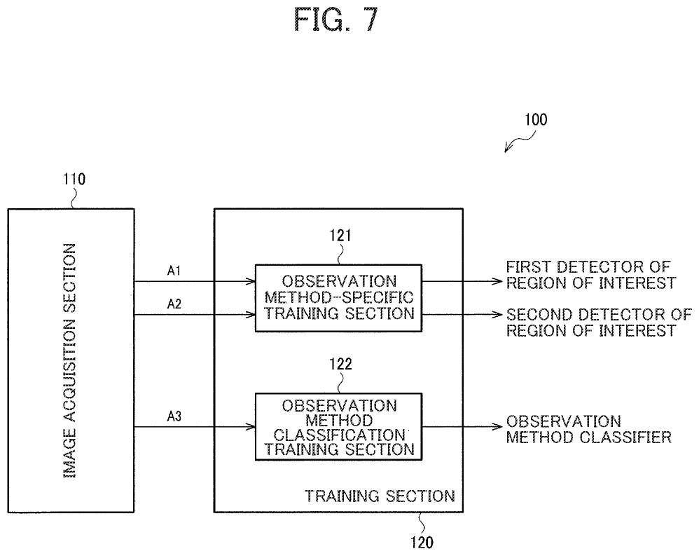

In addition, the training section 120 may be implemented by the following processor. The training device 100 includes a memory that stores information, and a processor that operates based on the information stored in the memory. The information is, for example, a program and various kinds of data or the like. The processor includes hardware. Note that various kinds of processors such as a central processing unit (CPU), a graphics processing unit (GPU), and a digital signal processor (DSP) can be used. The memory may be a semiconductor memory such as a static random-access memory (SRAM) and a dynamic random-access memory (DRAM). The memory may be a register. The memory may be a magnetic storage device such as a hard disk drive (HDD). The memory may be an optical storage device such as an optical disk device. For example, the memory stores a computer-readable instruction. The instruction is executed by the processor, whereby a function of each section of the training section 120 is implemented as a processing. Each section of the training section 120 is, for example, each section described later with reference to FIGS. 7, 13, and 14. The instruction mentioned herein may be an instruction of an instruction set that is included in a program, or may be an instruction that instructs a hardware circuit included in the processor to operate.

FIG. 3 illustrates a configuration example of the image processing system 200. The image processing system 200 includes an image acquisition section 210, a processing section 220, and a storage section 230.

The image acquisition section 210 acquires an in-vivo image captured by an imaging device of the endoscope system 300 as a processing target image. For example, the image acquisition section 210 is implemented as a communication interface that receives the in-vivo image via a network from the endoscope system 300. The network mentioned herein may be a private network such as an intranet, or may be a public telecommunication network such as the Internet. In addition, the network may be a wired network or a wireless network.

The processing section 220 operates in accordance with the trained model to perform the detection process of detecting the region of interest in the processing target image. Additionally, the processing section 220 determines information to be output based on a result of detection of the trained model. The processing section 220 comprises hardware including at least one of a digital signal processing circuit or an analog signal processing circuit. For example, the hardware can comprise one or more circuit devices mounted on a circuit board, or one or more circuit elements.

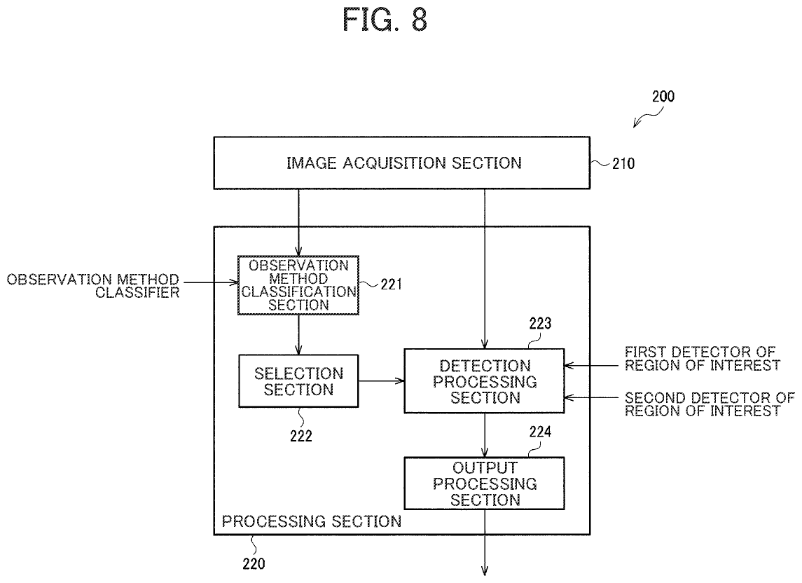

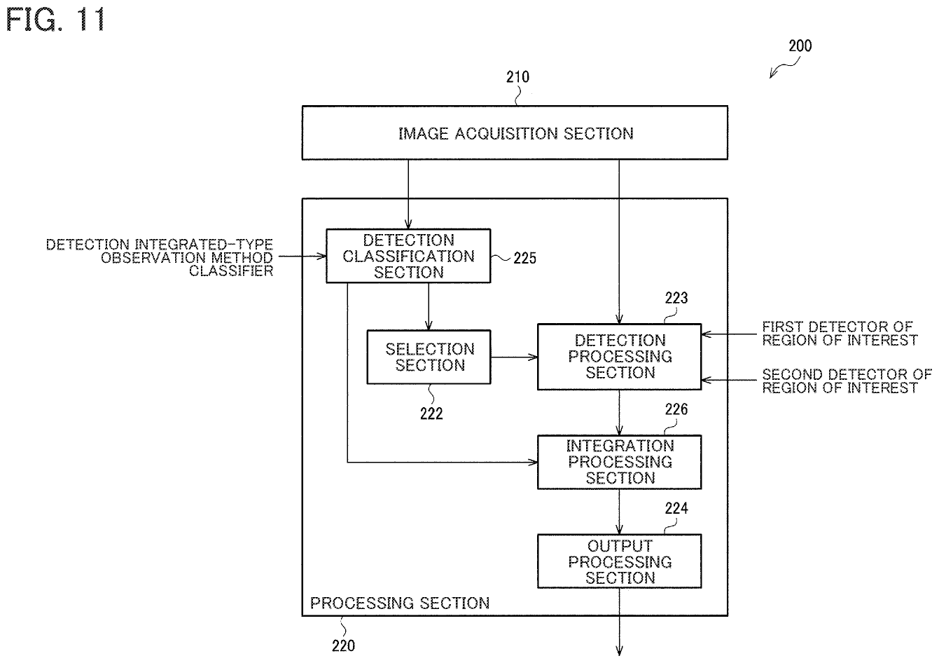

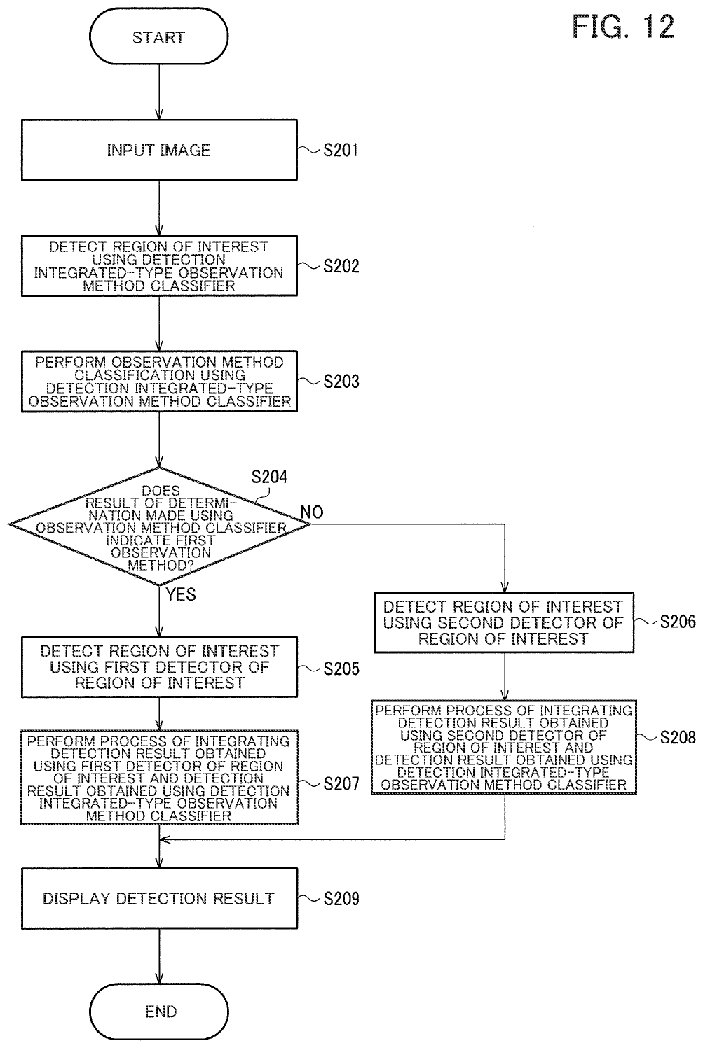

In addition, the processing section 220 may be implemented by the following processor. That is, the image processing system 200 includes a memory that stores information such as a program, and various kinds of data, and a processor that operates based on the information stored in the memory. The memory mentioned herein may be the storage section 230, or another different memory. Various kinds of processors such as a GPU can be used as the processor. The memory can be implemented in various manners such as a semiconductor memory, a resistor, a magnetic storage device, and an optical storage device. The memory stores a computer-readable instruction. The instruction is executed by the processor, whereby a function of each section of the processing section 220 is implemented. Each section of the processing section 220 is, for example, each section described later with reference to FIGS. 8 and 11.

The storage section 230 is a work area of the processing section 220 or the like, and the function thereof can be implemented by a semiconductor memory, a resistor, a magnetic storage device, or the like. The storage section 230 stores the processing target image acquired by the image acquisition section 210. Additionally, the storage section 230 stores information of the trained model generated by the training device 100.

FIG. 4 illustrates a configuration example of the endoscope system 300. The endoscope system 300 includes the insertion section 310, an external interface (I/F) section 320, the system control device 330, the display section 340, and a light source device 350.

The insertion section 310 is a portion whose distal end side is inserted into the body. The insertion section 310 includes an objective optical system 311, an image sensor 312, an actuator 313, an illumination lens 314, a light guide 315, and an auto focus (AF) start/end button 316.

The light guide 315 guides light emitted from a light source 352 to the distal end of the insertion section 310. The illumination lens 314 emits illumination light guided by the light guide 315 onto a subject. The objective optical system 311 receives reflected light from the subject and forms an image as a subject image. The objective optical system 311 includes a focus lens, and is capable of changing a position at which a subject image is formed in accordance with a position of the focus lens. The actuator 313 drives the focus lens based on an instruction from an AF control section 336. Note that AF is not essential, and the endoscope system 300 may have a configuration not including the AF control section 336.

The image sensor 312 receives light from the subject having passed through the objective optical system 311. The image sensor 312 may be a monochrome sensor, or may be an element having a color filter. The color filter may be a color filter in a well-known Bayer's arrangement, a complementary color filter, or another color filter. The complementary filter includes filters in respective colors of cyan, magenta, and yellow.

The AF start/end button 316 is an operation interface for a user to operate the start/end of AF. The external I/F section 320 is an interface by which the user performs an input operation to the endoscope system 300. The external I/F section 320 includes, for example, a button for setting an AF control mode, a button for setting an AF region, a button for adjusting an image processing parameter, and the like.

The system control device 330 performs image processing and control of the whole system. The system control device 330 includes an analog/digital (A/D) conversion section 331, a pre-processing section 332, a detection processing section 333, a post-processing section 334, a system control section 335, the AF control section 336, and a storage section 337.

The A/D conversion section 331 converts analog signals, which are sequentially output from the image sensor 312, to digital images, and sequentially outputs the digital images to the pre-processing section 332. The pre-processing section 332 performs various kinds of correction processes on in-vivo images sequentially output from the A/D conversion section 331, and sequentially outputs the in-vivo images to the detection processing section 333 and the AF control section 336. The correction processes include, for example, a white balance process, a noise reduction process, and the like.

The detection processing section 333, for example, performs a process of transmitting an image that has undergone the correction process and that is acquired from the pre-processing section 332 to the image processing system 200 arranged outside the endoscope system 300. The endoscope system 300 includes a communication section, which is not illustrated, and the detection processing section 333 performs communication control of the communication section. The communication section mentioned herein is a communication interface for transmitting an in-vivo image to the image processing system 200 via a given network. The detection processing section 333 performs communication control of the communication section to perform a process of receiving a detection result from the image processing system 200.

Alternatively, the system control device 330 may include the image processing system 200. In this case, the A/D conversion section 331 corresponds to the image acquisition section 210. The storage section 337 corresponds to the storage section 230. The pre-processing section 332, the detection processing section 333, the post-processing section 334, and the like correspond to the processing section 220. In this case, the detection processing section 333 operates in accordance with the information of the trained model stored in the storage section 337 to perform the detection process of detecting the region of interest targeting the in-vivo image serving as the processing target image. In a case where the trained model is the neural network, the detection processing section 333 performs a calculation process in a forward direction using weight determined by training on the processing target image serving as an input. The detection processing section 333 then outputs a detection result based on an output from an output layer.

The post-processing section 334 performs post-processing based on the detection result from the detection processing section 333, and outputs an image having undergone the post-processing to the display section 340. As the post-processing mentioned herein, various kinds of processing such as highlighting of a recognition target in the image and addition of information indicating the detection result can be assumed. For example, the post-processing section 334 superimposes a detection frame detected in the detection processing section 333 on the image output from the pre-processing section 332 to perform post-processing to generate a display image.

The system control section 335 is connected to each of the image sensor 312, the AF start/end button 316, the external I/F section 320, and the AF control section 336, and controls each section. Specifically, the system control section 335 inputs/outputs various kinds of control signals. The AF control section 336 uses images sequentially output from the pre-processing section 332 to perform AF control.

The display section 340 sequentially displays images output from the post-processing section 334. The display section 340 is, for example, a liquid crystal display, an electro-luminescence (EL) display, or the like. The light source device 350 includes the light source 352 that emits illumination light. The light source 352 may be a xenon light source, a light emitting diode (LED), or a laser light source. Alternatively, the light source 352 may be another light source, and a light emission method is not specifically limited.

Note that the light source device 350 is capable of emitting normal light and special light. For example, the light source device 350 includes a white light source and a rotary filter, and is capable of switching between normal light and special light based on rotation of the rotary filter. Alternatively, the light source device 350 may have a configuration of including a plurality of light sources such as a red LED, a green LED, a blue LED, a green narrow band light LED, and a blue narrow band light LED, to be capable of emitting a plurality of types of light having different wavelength bands. The light source device 350 turns on the red LED, the green LED, and the blue LED to emit normal light, and turns on the green narrow band light LED and the blue narrow band light LED to emit special light. Note that various kinds of configurations of the light source device that emits normal light and special light are known, and a wide range of them is applicable to the present embodiment.

### 1. First Embodiment

The following description is given of an example in which the first observation method is the normal light observation, and the second observation method is the special light observation. Note that the second observation method may be the pigment spray observation. That is, in the following description, the special light observation or the special light image can be replaced with the pigment spray observation and the pigment-sprayed image, respectively, where appropriate.

First, an outline of the machine learning is described. The machine learning using a neural network is described below. That is, the first detector of region of interest, the second detector of region of interest, and the observation method classifier described below are, for example, the trained model using the neural network. However, the method in accordance with the present embodiment is not limited thereto. In the present embodiment, for example, machine learning using another model such as a support vector machine (SVM) may be performed, and machine learning using a method that has developed from various methods such as the neural network and the SVM may be performed.

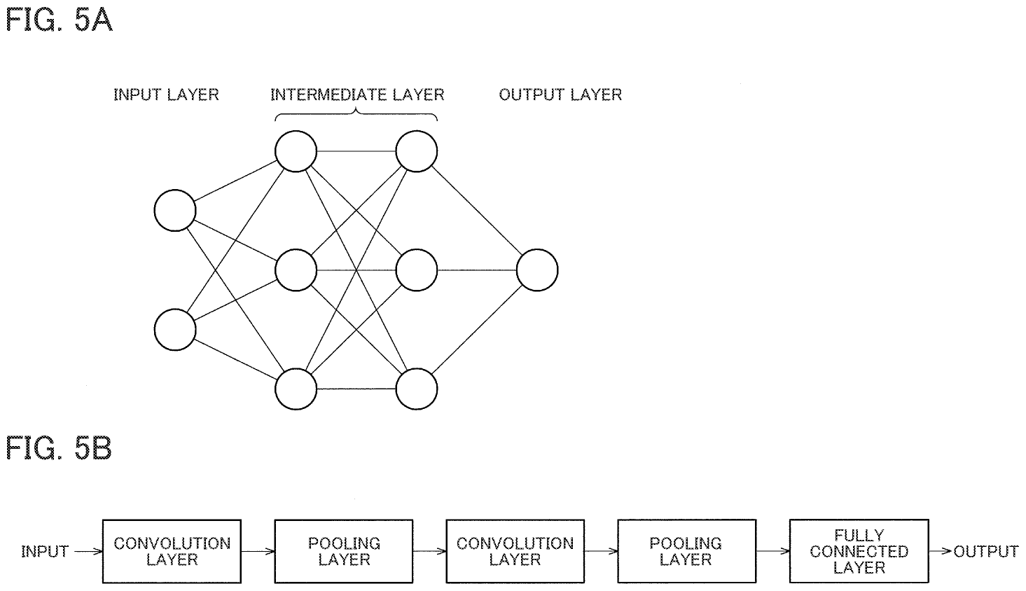

FIG. 5A is a schematic diagram for describing the neural network. The neural network includes an input layer that takes input data, an intermediate layer that executes calculation based on an output from the input layer, and an output layer that outputs data based on an output from the intermediate layer. While FIG. 5A exemplifies a network having the intermediate layer comprising two layers, the intermediate layer may comprise one layer, or three or more layers. In addition, the number of nodes (neurons) included in each layer is not limited to that in the example of FIG. 5A, and can be modified in various manners. Note that in consideration of accuracy, the training in accordance with the present embodiment is preferably performed using deep learning using a multi-layer neural network. The multi-layer mentioned herein means four or more layers in a more limited sense.

As illustrated in FIG. 5A, a node included in a given layer is connected to a node in an adjacent layer. A weight coefficient is assigned between connected nodes. Each node multiplies an output from a node in a former stage by the weight coefficient and obtains a total value of results of multiplication. Furthermore, each node adds a bias to the total value and applies an activation function to a result of addition to obtain an output from the node. This process is sequentially executed from the input layer to the output layer, whereby an output from the neural network is obtained. Note that as the activation function, various functions such as a sigmoid function and a rectified linear unit (ReLU) function are known, and a wide range of these functions can be applied in the present embodiment.

The training in the neural network is a process of determining an appropriate weight coefficient. The weight coefficient mentioned herein includes a bias. Specifically, the training device 100 inputs input data out of training data to the neural network and performs calculation in the forward direction using the weight coefficient at this time to obtain an output. The training section 120 of the training device 100 performs calculation to obtain an error function based on the output and ground truth (correct data) out of the training data. The training section 120 updates the weight coefficient to make the error function smaller. In updating the weight coefficient, for example, backpropagation to update the weight coefficient from the output layer to the input layer can be utilized.

The neural network may be, for example, a convolutional neural network (CNN). FIG. 5B is a schematic diagram for describing the CNN. The CNN includes a convolution layer that performs convolution calculation and a pooling layer. The convolution layer is a layer that performs a filter process. The pooling layer is a layer that reduces a size in a vertical direction and a size in a lateral direction to perform pooling calculation. In the example illustrated in FIG. 5B, the CNN is a network that causes each of the convolution layer and the pooling layer to perform calculation a plurality of times, thereafter causes a fully connected layer to perform calculation, and thereby obtain an output. The fully connected layer is a layer that performs a calculation process in a case where all nodes included in the former layer are connected to corresponding nodes in the given layer, and the calculation process corresponds to calculation in each layer described above with reference to FIG. 5A. Note that although not illustrated in FIG. 5B, the calculation process with the activation function is also performed in the CNN. Various kinds of configurations of the CNN have been known, and a wide range of these configurations are applicable to the present embodiment. For example, a known Region Proposal Network (RPN) or the like can be utilized as the CNN in accordance with the present embodiment.

In a case where the CNN is used, a procedure of processing is similar to that illustrated in FIG. 5A. That is, the training device 100 inputs input data, out of the training data, to the CNN, and performs a filter process or pooling calculation using filter characteristics at that time to obtain an output. The training device 100 calculates the error function based on the output and the ground truth, and updates the weight coefficient including the filter characteristics to make the error function smaller. For example, the backpropagation can be utilized also when the weight coefficient of the CNN is updated.

Subsequently, the machine learning in accordance with the present embodiment is described. The detection process of a region of interest executed by the image processing system 200 is, specifically, a process of detecting at least one of whether the region of interest is present in each first training image, and, if any, a position, a size, and a shape of the region of interest.

For example, the detection process is a process of obtaining information that identifies a rectangular frame region surrounding the region of interest and a detection score indicating a probability in the frame region. The frame region is hereinafter referred to as a detection frame. The information that identifies the detection frame is, for example, four numeric values comprising a coordinate value of an upper left end point of the detection frame on an abscissa axis, a coordinate value of the end point on an ordinate axis, a length of the detection frame in an abscissa axis direction, and a length of the detection frame in an ordinate axis direction. Since an aspect ratio of the detection frame changes with change of the shape of the region of interest, the detection frame corresponds to information indicating not only whether the region of interest is present, and, if any, a position and a size, but also a shape of the region of interest, but also the shape of the region of interest. Note that widely known segmentation may be used in the detection process in accordance with the present embodiment. In this case, with respect to each pixel in the image, information indicating whether or not the pixel is the region of interest, for example, information indicating whether or not the pixel corresponds to a polyp is output. In this case, it is possible to identify the shape of the region of interest in a more detailed manner.

FIG. 7 illustrates a configuration example of the training device 100 in accordance with a first embodiment. The training section 120 of the training device 100 includes an observation method-specific training section 121 and an observation method classification training section 122. The observation method-specific training section 121 acquires an image group A1 from the image acquisition section 110, performs machine learning based on the image group A1, and thereby generates the first detector of region of interest. The observation method-specific training section 121 acquires an image group A2 from the image acquisition section 110, performs machine learning based on the image group A2, and thereby generates the second detector of region of interest. That is, the observation method-specific training section 121 generates a plurality of trained models based on a plurality of different image groups.

A training process executed in the observation method-specific training section 121 is a training process for generating a trained model dedicated to either the normal light image or the special light image. That is, the image group A1 includes a training image in which the normal light image is provided with detection data, which is information regarding at least one of whether the region of interest is present, and, if any, a position, a size, and a shape of the region of interest. The image group A1 does not include a training image in which the special light image is provided with the detection data, or even if the image group A1 includes the training image, the number of training images is sufficiently smaller than the number of normal light images.

For example, the detection data is mask data in which a polyp region serving as a detection target and a background region are filled with different colors. Alternatively, the detection data may be information for identifying a detection frame surrounding a polyp. For example, the training image included in the image group A1 may be data in which the polyp region in the normal light image is surrounded by a rectangular frame, and the rectangular frame is provided with a label of “POLYP”, and the other region is provided with a label of “NORMAL”. Note that the detection frame is not limited to the rectangular frame. The detection frame is only required to surround the vicinity of the polyp region, and may be an ellipsoidal frame or the like.

The image group A2 includes a training image in which the special light image is provided with the detection data. The image group A2 does not include a training image in which the normal light image is provided with the detection data, or even if the image group A2 includes the training image, the number of training images is sufficiently smaller than the number of special light images. The detection data is similar to that in the image group A1, and may be mask data or information that identifies the detection frame.

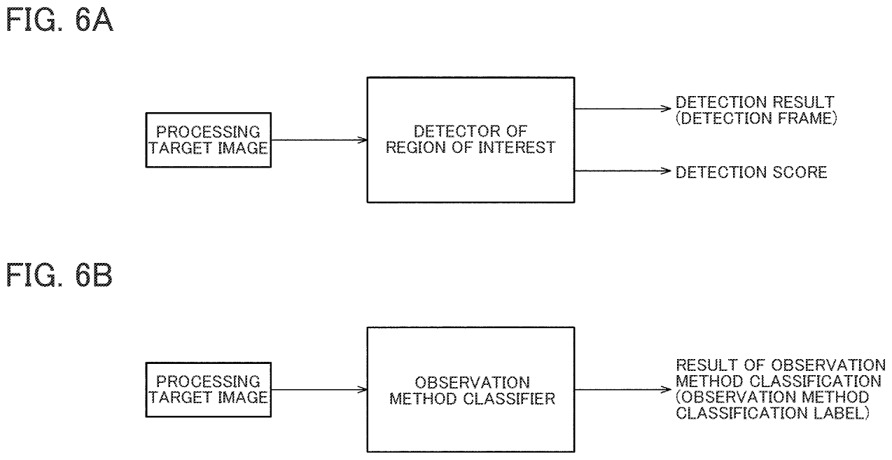

FIG. 6A is a diagram for describing an input to and an output from each of the first detector of region of interest and the second detector of region of interest. Each of the first detector of region of interest and the second detector of region of interest accepts a processing target image as the input, performs processing on the processing target image, and thereby outputs information indicating a detection result. The observation method-specific training section 121 performs machine learning of a model including an input layer that takes an input image, an intermediate layer, and an output layer that outputs a detection result. For example, each of the first detector of region of interest and the second detector of region of interest is a CNN for detecting an object, such as an RPN, a Faster Region-Based Convolutional Neural Networks (Faster R-CNN), and You Only Look Once (YOLO).

Specifically, the observation method-specific training section 121 performs calculation in the forward direction based on a present weight coefficient with the training image included in the image group A1 serving as the input to the neural network. The observation method-specific training section 121 calculates, as an error function, an error between the output from the output layer and the detection data serving as ground truth, and performs a process of updating the weight coefficient so as to make the error function smaller. This is the process based on one training image, and the observation method-specific training section 121 repeats the above-mentioned processing to perform training of the weight coefficient of the first detector of region of interest. Note that the updating of the weight coefficient is not limited to the one performed on an image-by-image basis, and batch training or the like may be used.

Similarly, the observation method-specific training section 121 performs calculation in the forward direction based on the present weight coefficient with the training image included in the image group A2 serving as the input to the neural network. The observation method-specific training section 121 calculates, as the error function, an error between the output from the output layer and the detection data serving as the ground truth, and performs the process of updating the weight coefficient so as to make the error function smaller. The observation method-specific training section 121 repeats the above-mentioned processing to perform training of the weight coefficient of the second detector of region of interest.

An image group A3 is an image group including a training image in which the normal light image is provided with observation method data, which is information that identifies an observation method, as the ground truth, and a training image in which the special light image is provided with the observation method data. The observation method data is, for example, a label indicating either the normal light image or the special light image.

FIG. 6B is a diagram for describing an input to and an output from the observation method classifier. The observation method classifier accepts the processing target image as the input, performs processing on the processing target image, and thereby outputs information indicating a result of observation method classification.

The observation method classification training section 122 performs machine learning of the model including an input layer that takes an input image, and an output layer that outputs the result of observation method classification. The observation method classifier is, for example, a CNN for classifying an image such as a Visual Geometry Group-16 (VGG-16) and a Residual Neural Network (ResNet). The observation method classification training section 122 performs calculation in the forward direction based on the present weight coefficient with the training image included in the image group A3 serving as the input to the neural network. The observation method-specific training section 121 calculates, as the error function, the error between the output from the output layer and the observation data serving as the ground truth, and performs the process of updating the weight coefficient so as to make the error function smaller. The observation method classification training section 122 repeats the above-mentioned process to perform training of the weight coefficient of the observation method classifier.

Note that the output from the output layer in the observation method classifier includes data indicating a probability that the input image is the normal light image captured in the normal light observation, and data indicating a probability that the input image is the special light image captured in the special light observation. For example, in a case where the output layer of the observation method classifier is a known softmax layer, the output layer outputs two pieces of probability data, a total value of which is 1. In a case where the label serving as the ground truth indicates the normal light image, the error function is obtained using data in which the probability data for the normal light image is 1 and the probability data for the special light image is 0 as the ground truth. The observation method classifier is capable of outputting an observation method classification label serving as the result of observation method classification, and an observation method classification score indicating a probability of the observation method classification label. The observation method classification label is a label indicating an observation method that maximizes the probability data, and is, for example, a label indicating either the normal light observation or the special light observation. The observation method classification score is probability data corresponding to the observation method classification label. The observation method classification score is not illustrated in FIG. 6B.

FIG. 8 illustrates a configuration example of the image processing system 200 in accordance with the first embodiment. The processing section 220 of the image processing system 200 includes an observation method classification section 221, a selection section 222, a detection processing section 223, and an output processing section 224. The observation method classification section 221 performs an observation method classification process based on the observation method classifier. The selection section 222 selects a detector of region of interest based on a result of the observation method classification process. The detection processing section 223 performs a detection process using at least one of the first detector of region of interest or the second detector of region of interest. The output processing section 224 performs an output process based on a detection result.

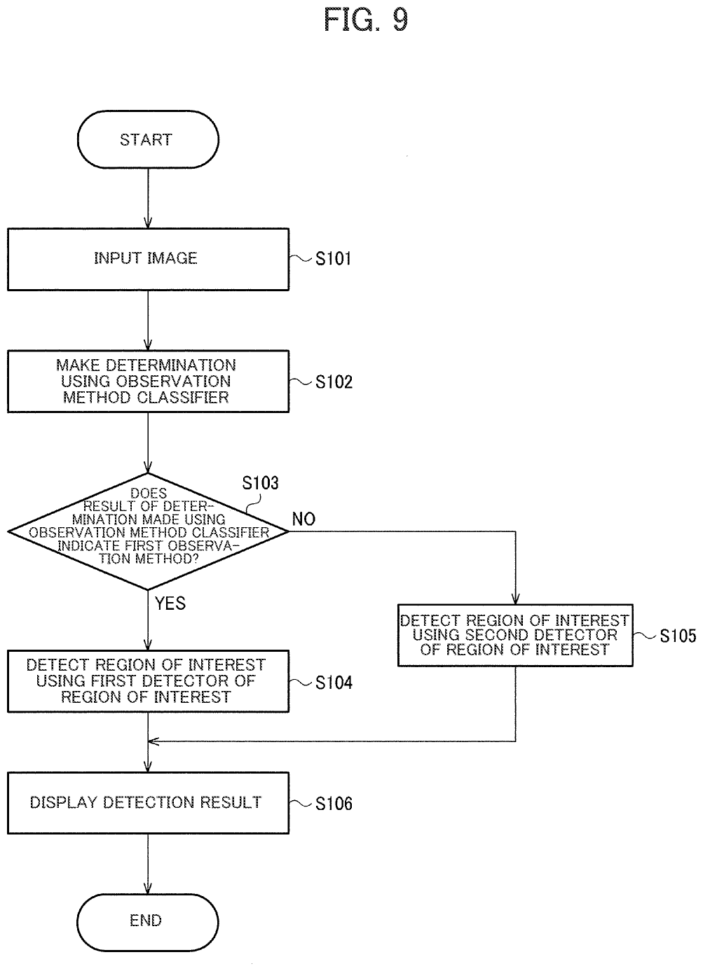

FIG. 9 is a flowchart describing processing of the image processing system 200 in accordance with the first embodiment. First, in step S101, the image acquisition section 210 acquires an in-vivo image captured by the endoscope imaging device as a processing target image.

In step S102, the observation method classification section 221 performs the observation method classification process of determining whether the processing target image is the normal light image or the special light image. For example, the observation method classification section 221 inputs the processing target image acquired by the image acquisition section 210 to the observation method classifier, and thereby acquires probability data indicating a probability that the processing target image is the normal light image and probability data indicating a probability that the processing target image is the special light image. The observation method classification section 221 performs the observation method classification process based on a magnitude relationship between the two pieces of probability data.

In step S103, the selection section 222 selects a detector of region of interest based on a result of observation method classification. In a case where the result of observation method classification indicating that the processing target image is the normal light image is acquired, the selection section 222 selects the first detector of region of interest. In a case where the result of observation method classification indicating that the processing target image is the special light image is acquired, the selection section 222 selects the second detector of region of interest. The selection section 222 transmits a selection result to the detection processing section 223.

In a case where the selection section 222 selects the first detector of region of interest, in step S104, the detection processing section 223 performs a detection process of detecting the region of interest using the first detector of region of interest. Specifically, the detection processing section 223 inputs the processing target image to the first detector of region of interest, and thereby acquires information regarding a predetermined number of detection frames in the processing target image and a detection score associated with each detection frame. The detection result in the present embodiment indicates, for example, the detection frame, and the detection score indicates a probability of the detection result.

In a case where the selection section 222 selects the second detector of region of interest, in step S105, the detection processing section 223 performs the detection process of detecting the region of interest using the second detector of region of interest. Specifically, the detection processing section 223 inputs the processing target image to the second detector of region of interest, and thereby acquires the detection frame and the detection score.

In step S106, the output processing section 224 outputs the detection result acquired in step S104 or step S105. For example, the output processing section 224 performs a process of comparing the detection score and a given detection threshold. In a case where the detection score in the given detection frame is less than the detection threshold, information regarding the detection frame has low reliability, and is thus excluded from an output target.

In a case where the image processing system 200 is included in the endoscope system 300, the processing in step S106 is, for example, a process of generating a display image, and a process of displaying the display image on the display section 340. In a case where the image processing system 200 and the endoscope system 300 are arranged as individual devices, the above-mentioned processing is, for example, a process of transmitting the display image to the endoscope system 300. Alternatively, the above-described processing may be a process of transmitting the information indicating the detection frame to the endoscope system 300. In this case, each of the process of generating the display image and the display control is executed in the endoscope system 300.

As described above, the image processing system 200 in accordance with the present embodiment includes the image acquisition section 210 that acquires the processing target image, and the processing section 220 that performs the process of outputting the detection result, which is a result of detecting the region of interest in the processing target image. As illustrated in FIG. 8 and described in steps S102 and S103 in FIG. 9, the processing section 220 performs the classification process of classifying an observation method of a subject where the processing target image is captured to one of the plurality of observation methods including the first observation method and the second observation method based on the observation method classifier, and the selection process of selecting one of the plurality of detectors of region of interest including the first detector of region of interest and the second detector of region of interest based on the classification result from the observation method classifier. Note that in the first embodiment, the plurality of observation methods are the two observation methods comprising the first observation method and the second observation method. The plurality of detectors of region of interest are the two detectors of region of interest comprising the first detector of region of interest and the second detector of region of interest. Hence, the processing section 220 performs the observation method classification process of classifying the observation method for capturing the processing target image to either the first observation method or the second observation method based on the observation method classifier, and the selection process of selecting the first detector of region of interest or the second detector of region of interest based on the classification result from the observation method classifier. However, as described later in a third embodiment, there may be three or more observation methods. There may be also three or more detectors of region of interest. In a case where an observation method mixed-type detector of region of interest, such as CNN_AB which will be described later, is used, the number of detectors of region of interest may be more than the number of observation methods, and the number of detectors of region of interest selected by the one-time selection process may be two or more.

The processing section 220 outputs, when the first detector of region of interest is selected in the selection process, the detection result in which the region of interest is detected from the processing target image classified to the first observation method based on the first detector of region of interest. The processing section 220 outputs, when the second detector of region of interest is selected in the selection process, the detection result in which the region of interest is detected from the processing target image classified to the second observation method based on the second detector of region of interest.

In the method in accordance with the present embodiment, in a case where different observation methods are assumed, a detector of region of interest appropriate for each observation method is created. On the premise of the above, selecting the appropriate detector of region of interest based on the result of classifying the observation method when the processing target image is captured enables execution of the detection process with high accuracy, regardless of the observation method for the processing target image. Note that the above description has been given of the example of performing either the detection process using the first detector of region of interest or the detection process using the second detector of region of interest, but the flow of processing is not limited thereto. For example, the detection processing section 223 may be configured to perform both the detection process using the first detector of region of interest and the detection process using the second detector of region of interest, and thereafter transmit the detection result of either one of the detection processes to the output processing section 224 based on the result of observation method classification.

Note that the respective processing based on the observation method classifier, the first detector of region of interest, and the second detector of region of interest is implemented by operations of the processing section 220 in accordance with an instruction from the trained model. Calculation in accordance with the trained model in the processing section 220, that is, calculation for outputting output data based on input data may be executed by software, or hardware. In other words, product-sum calculation executed at each node in FIG. 5A, a filter process executed in the convolution layer of the CNN, or the like may be executed by software. Alternatively, the above-mentioned calculation may be executed by a circuit device such as a FPGA circuit. Still alternatively, the above-mentioned calculation may be executed by software and hardware in combination. In this manner, operations of the processing section 220 in accordance with an instruction from the trained model can be implemented in various manners. For example, the trained model includes an inference algorithm, and a parameter used in the inference algorithm. The inference algorithm is an algorithm that performs filter calculation or the like based on the input data. The parameter is a parameter acquired by a training process, and is, for example, a weight coefficient. In this case, both the inference algorithm and the parameter are stored in the storage section 230, and the processing section 220 may read out the inference algorithm and the parameter and thereby perform the inference process with software. Alternatively, the inference algorithm may be implemented by the FPGA circuit or the like, and the storage section 230 may store the parameter. Still alternatively, the inference algorithm including the parameter may be implemented by the FPGA circuit or the like. In this case, the storage section 230 that stores information of the trained model is, for example, a built-in memory of the FPGA circuit.

In addition, the processing target image in accordance with the present embodiment is an in-vivo image captured by the endoscope imaging device. The endoscope imaging device mentioned herein is an imaging device that is arranged in the endoscope system 300 and that is capable of outputting a result of formation of a subject image corresponding to the living body, and corresponds to the image sensor 312 in a more limited sense.

The first observation method is an observation method using normal light as illumination light, and the second observation method is an observation method using special light as illumination light. With this configuration, even in a case where the observation method is changed by switching of illumination light between normal light and special light, it is possible to prevent a decrease in detection accuracy due to the change.

The first observation method is an observation method using normal light as illumination light, and the second observation method may be an observation method in which a pigment has been sprayed onto the subject. With this configuration, even in a case where the observation method is changed by spray of a color material onto the subject, it is possible to prevent a decrease in detection accuracy due to the change.

Since the special light observation and the pigment spray observation can enhance visibility of a specific subject as compared with the normal light observation, a combined use thereof with the normal light observation provides many advantages. In accordance with the method of the present embodiment, the special light observation or the pigment spray observation enables achievement of both provision of an image with high visibility for a user and maintenance of accuracy of detection by the detector of region of interest.

The first detector of region of interest is a trained model acquired by machine learning performed based on a plurality of first training images captured in the first observation method, and detection data regarding at least one of whether the region of interest is present in each first training image, and, if any, a position, a size, and a shape of the region of interest. The second detector of region of interest is a trained model acquired by machine learning performed based on a plurality of second training images captured in the second observation method, and detection data regarding at least one of whether the region of interest is present in each second training image, and, if any, a position, a size, and a shape of the region of interest.

With this configuration, it is possible to match an observation method for the training image used in a training stage and an observation method for the processing target image serving as the input in an inference stage with each other. Hence, it is possible to use the trained model desirable for the detection process targeting the image captured in the first observation method as the first detector of region of interest. Similarly, it is possible to use the trained model desirable for the detection process targeting the image captured in the second observation method as the second detector of region of interest.

At least one of the observation method classifier, the first detector of region of interest, or the second detector of region of interest in accordance with the present embodiment may comprise a CNN. For example, each of the observation method classifier, the first detector of region of interest, and the second detector of region of interest may be the CNN. This enables execution of the detection process with an image as an input efficiently with high accuracy. Note that part of the observation method classifier, the first detector of region of interest, and the second detector of region of interest may have a configuration other than that of the CNN. Note that the CNN is not an essential configuration, and a possibility that each of the observation method classifier, the first detector of region of interest, and the second detector of region of interest has a configuration other than that of the CNN is not precluded.

Additionally, the method in accordance with the present embodiment can be applied to the endoscope system 300. The endoscope system 300 includes an imaging section that captures an in-vivo image, an image acquisition section that acquires the in-vivo image as a processing target image, and a processing section that performs processing on the processing target image. As described above, the imaging section in this case is, for example, the image sensor 312. The image acquisition section is, for example, the A/D conversion section 331. The processing section corresponds to the pre-processing section 332, the detection processing section 333, the post-processing section 334, and the like. Note that the image acquisition section can be assumed to correspond to the A/D conversion section 331 and the pre-processing section 332, and a specific configuration thereof can be modified in various manners.

The processing section of the endoscope system 300 performs the classification process of classifying the observation method when the processing target image is captured to one of the plurality of observation methods including the first observation method and the second observation method based on the observation method classifier, and the selection process of selecting one of the plurality of detectors of region of interest including the first detector of region of interest and the second detector of region of interest based on the classification result from the observation method classifier. The processing section outputs, when the first detector of region of interest is selected in the selection process, the detection result in which the region of interest is detected from the processing target image classified to the first observation method based on the first detector of region of interest. The processing section outputs, when the second detector of region of interest is selected in the selection process, the detection result in which the region of interest is detected from the processing target image classified to the second observation method based on the second detector of region of interest.

This enables execution of the detection process targeting the in-vivo image with high accuracy regardless of an observation method in the endoscope system 300 that captures an in-vivo image. Presenting the detection result to a doctor on the display section 340 or the like enables provision of appropriate support for the doctor's diagnosis or the like.

The processing executed by the image processing system 200 in accordance with the present embodiment may be implemented as an image processing method. The image processing method in accordance with the present embodiment includes acquisition of the processing target image, execution of the classification process of classifying the observation method when the processing target image is captured to one of the plurality of observation methods including the first observation method and the second observation method based on the observation method classifier, and execution of the selection process of selecting one of the plurality of detectors of region of interest including the first detector of region of interest and the second detector of region of interest based on the classification result from the observation method classifier. Furthermore, the image processing method includes, when the first detector of region of interest is selected in the selection process, outputting of the detection result in which the region of interest is detected from the processing target image classified to the first observation method based on the first detector of region of interest. In addition, when the second detector of region of interest is selected in the selection process, the image processing method includes outputting of the detection result in which the region of interest is detected from the processing target image classified to the second observation method based on the second detector of region of interest.

### 3. Second Embodiment

In the first embodiment, the example in which the observation method classifier executes only the observation method classification process has been described. However, the observation method classifier may execute a process of detecting the region of interest in addition to the observation method classification process. Note that also in the second embodiment, a description will be given of an example in which the first observation method is the normal light observation and the second observation method is the special light observation, but the second observation method may be the pigment spray observation.

A configuration of the training device 100 is similar to that illustrated in FIG. 7, and the training section 120 includes the observation method-specific training section 121 that generates the first detector of region of interest and the second detector of region of interest, and the observation method classification training section 122 that generates the observation method classifier. However, in the present embodiment, a configuration of the observation method classifier and image groups used for machine learning for generating the observation method classifier are different. Note that the observation method classifier in accordance with the second embodiment is also referred to as the detection integrated-type observation method classifier for distinguishing the observation method classifier in accordance with the second embodiment from that in accordance with the first embodiment.

The detection integrated-type observation method classifier employs, for example, a configuration in which a CNN for detecting the region of interest and a CNN for classifying an observation method share a feature extraction layer that extracts features while repeating a convolution process, a pooling process, and a non-linear activation process, and outputs are divided into an output as a detection result and an output as a result of observation method classification.

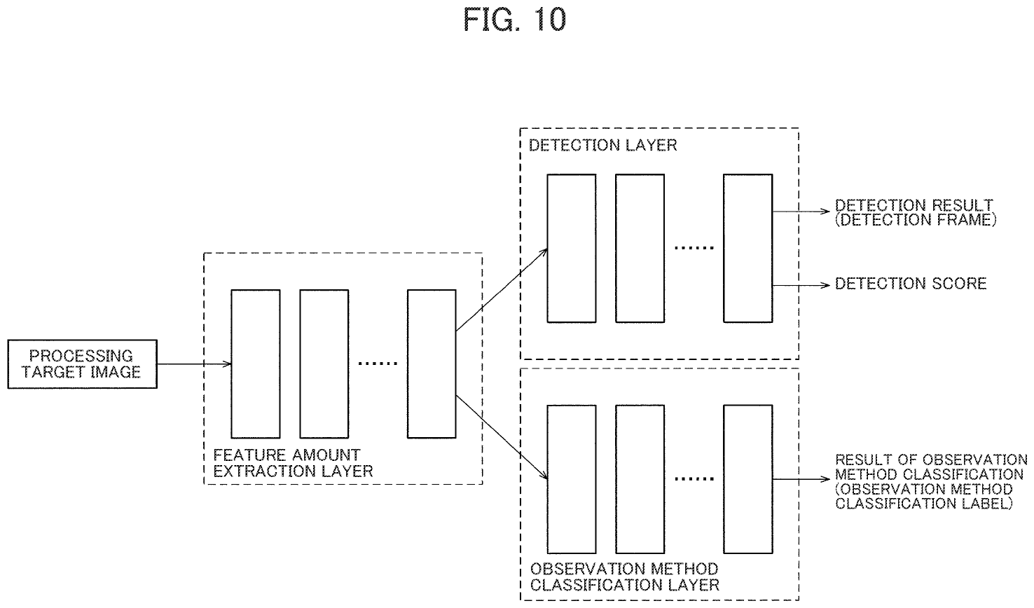

FIG. 10 is a diagram illustrating a configuration of a neural network of the observation method classifier in accordance with the second embodiment. As illustrated in FIG. 10, the CNN as the detection integrated-type observation method classifier includes a feature amount extraction layer, a detection layer, and an observation method classification layer. Each rectangular region in FIG. 10 represents a layer that performs some kind of calculation in the convolution layer, the pooling layer, the fully connected layer, or the like. Note that the configuration of the CNN is not limited to that illustrated in FIG. 10, and may be modified in various manners.

The feature amount extraction layer accepts the processing target image as an input, performs calculation including convolution calculation, and thereby outputs a feature amount. The detection layer uses the feature amount output from the feature amount extraction layer as an input, and outputs information indicating a detection result. The observation method classification layer uses the feature amount output from the feature amount extraction layer as an input, and outputs information indicating a result of observation method classification. The training device 100 executes a training process to determine a weight coefficient in each of the feature amount extraction layer, the detection layer, and the observation method classification layer.

The observation method classification training section 122 in accordance with the present embodiment performs a process based on an image group including a training image in which the normal light image is provided with detection data and observation method data as ground truth, and a training image in which the special light image is provided with the detection data and the observation method data, and thereby generates the detection integrated-type observation method classifier.

Specifically, the observation method classification training section 122 performs calculation in the forward direction based on a present weight coefficient with the normal light image and the special light image included in the image group serving as an input in the neural network illustrated in FIG. 10. The observation method classification training section 122 calculates, as an error function, an error between a result obtained by the calculation in the forward direction and the ground truth, and performs a process of updating the weight coefficient so as to make the error function smaller. For example, the observation method classification training section 122 obtains, as the error function, a weighted sum of an error between an output from the detection layer and the detection data and an error between an output from the observation method classification layer and the observation method data. That is, in training of the detection integrated-type observation method classifier, the weight coefficient of the feature amount extraction layer, the weight coefficient of the detection layer, the weight coefficient of the observation method classification layer out of the neural network illustrated in FIG. 10 each serve as a target of training.

FIG. 11 illustrates a configuration example of the image processing system 200 in accordance with the second embodiment. The processing section 220 of the image processing system 200 includes a detection classification section 225, the selection section 222, the detection processing section 223, an integration processing section 226, and the output processing section 224. The detection classification section 225 outputs a detection result and a result of observation method classification based on the detection integrated-type observation method classifier generated by the training device 100. The selection section 222 and the detection processing section 223 are similar to those of the first embodiment. The integration processing section 226 performs an integration process of integrating a detection result from the detection classification section 225 and a detection result from the detection processing section 223. The output processing section 224 performs an output process based on a result of the integration process.

FIG. 12 is a flowchart describing processing of the image processing system 200 in accordance with the second embodiment. First, in step S201, the image acquisition section 210 acquires an in-vivo image captured by the endoscope imaging device as a processing target image.

In steps S202 and S203, the detection classification section 225 performs calculation in the forward direction with the processing target image acquired by the image acquisition section 210 serving as the input to the detection integrated-type observation method classifier. In processing in step S202 and S203, the detection classification section 225 acquires information indicating a detection result from the detection layer and information indicating a result of observation method classification from the observation method classification layer. Specifically, the detection classification section 225 acquires a detection frame and a detection score in the processing in step S202. Additionally, in the processing in step S203, the detection classification section 225 acquires probability data indicating a probability that the processing target image is the normal light image, and probability data indicating a probability that the processing target image is the special light image. The detection classification section 225 performs an observation method classification process based on a magnitude relationship between the two pieces of probability data.

Processing in steps S204 to S206 is similar to the processing in steps S103 to S105 described in FIG. 9. That is, in step S204, the selection section 222 selects a detector of region of interest based on the result of the observation method classification. In a case where the result of the observation method classification indicating that the processing target image is the normal light image is acquired, the selection section 222 selects the first detector of region of interest. In a case where the result of the observation method classification indicating that the processing target image is the special light image is acquired, the selection section 222 selects the second detector of region of interest.

In a case where the selection section 222 selects the first detector of region of interest, in step S205, the detection processing section 223 performs a detection process of detecting the region of interest using the first detector of region of interest, and thereby obtains a detection result. In a case where the selection section 222 selects the second detector of region of interest, in step S206, the detection processing section 223 performs a detection process of detecting the region of interest using the second detector of region of interest, and thereby obtains a detection result.

In step S207 after the processing in step S205, the integration processing section 226 performs an integration process of integrating the detection result obtained using the detection-integrated-type observation method classifier and the detection result obtained using the first detector of region of interest. Even in detection results of the same region of interest, the position of the detection frame, the size of the detection frame, or the like that is output using the detection integrated-type observation method classifier and the position of the detection frame, the size of the detection frame, or the like that is output using the first detector of region of interest are not necessarily matched with each other. At this time, if both the detection result using the detection integrated-type observation method classifier and the detection result using the first detector of region of interest are output, a plurality of pieces of information that are different with respect to one region of interest are displayed, resulting in confusion for a user.

To address this, the integration processing section 226 determines whether the detection frame detected by the detection integrated-type observation method classifier and the detection frame detected by the first detector of region of interest are regions corresponding to the same region of interest. For example, the integration processing section 226 calculates Intersection over Union (IoU) indicating a degree of overlap between the detection frames. In a case where the IoU is a threshold or greater, the integration processing section 226 determines that the two detection frames correspond to the same region of interest. Since the IoU is a known technique, a detailed description thereof is omitted. The threshold of the IoU is, for example, about 0.5, but a specific numeric value can be modified in various manners.

In a case of determining that the two detection frames correspond to the same region of interest, the integration processing section 226 may select a detection frame having a high detection score as the detection frame corresponding to the region of interest, or may set a new detection frame based on the two detection frames. Alternatively, the integration processing section 226 may select a higher detection score out of two detection scores as a detection score associated with the detection frame, or may use a weighted sum of the two detection scores.

Meanwhile, in step S208 after the processing in step S206, the integration processing section 226 performs an integration process of integrating the detection result obtained using the detection integrated-type observation method classifier and the detection result obtained using the second detector of region of interest. The flow of the integration process is similar to that in step S207.

As a result of the integration process in step S206 or S208, one detection result is acquired with respect to one region of interest. That is, the output of the integration process is information indicating detection frames whose number corresponds to the number of regions of interest in the processing target image, and a detection score in each detection frame. Hence, the output processing section 224 performs an output process that is similar to that in the first embodiment.

As described above, the processing section 220 of the image processing system 200 in accordance with the present embodiment performs the process of detecting the region of interest from the processing target image based on the observation method classifier.

This allows the observation method classifier to also serve as the detector of region of interest. The observation method classifier includes both a training image that is captured in the first observation method and a training image that is captured in the second observation method, in order to execute observation method classification. For example, the detection integrated-type observation method classifier includes both the normal light image and the special light image as training images. As a result, the detection integrated-type observation method classifier can execute a versatile detection process that can be applied to each of the case where the processing target image is the normal light image and the case where the processing target image is the special light image. That is, in accordance with the method of the present embodiment, an effective configuration enables acquisition of a detection result with high accuracy.

The processing section 220 performs, when the first detector of region of interest is selected in the selection process, the integration process of integrating the detection result of the region of interest based on the first detector of region of interest and the detection result of the region of interest based on the observation method classifier. In addition, the processing section 220 performs, when the second detector of region of interest is selected in the selection process, the integration process of integrating the detection result of the region of interest based on the second detector of region of interest and the detection result of the region of interest based on the observation method classifier.

The integration process is, for example, the process of determining the detection frame corresponding to the region of interest based on the two detection frames and the process of determining the detection score to be associated with the detection frame based on the two detection scores, as described above. However, the integration process in accordance with the present embodiment is only required to be a process of determining one detection result with respect to one region of interest based on two detection results, and specific processing contents and a format of information to be output as a detection result can be modified in various manners.

In this manner, integrating a plurality of detection results enables acquisition of a detection result with higher accuracy. For example, in a case where data is poorly balanced between the two observation methods, the first detector of region of interest having undergone training dedicated to the first observation method or the second detector of region of interest having undergone training dedicated to the second observation method exhibits relatively higher accuracy. On the other hand, in a case where data is well balanced between the two observation methods, the detection integrated-type observation method classifier that includes images captured both in the first observation method and the second observation method exhibits relatively higher accuracy. The balancing of data represents a ratio of the number of images in an image group used for training.

The balancing of data in the observation method changes due to various factors such as an operational status of the endoscope system serving as a source for collecting data and a status of provision of ground truth. Additionally, in a case where collection is continuously performed, it is assumed that the balancing of data changes with time. While the training device 100 can adjust the balancing of data or change the training process in accordance with the balancing of data, a load of the training process becomes heavier. While the inference process in the image processing system 200 can be changed in consideration of the balancing of data in a training stage, it is necessary to acquire information regarding the balancing of data or to branch processing in accordance with the balancing of data, leading to a heavy load. In this regard, performing the integration process as described above enables presentation of a result with high accuracy in a complementary manner regardless of the balancing of data without increasing a processing load.

The processing section 220 performs at least one of a process of outputting a first score indicating a probability that a region detected as the region of interest from the processing target image based on the first detector of region of interest, or a process of outputting a second score indicating a probability that a region detected as the region of interest from the processing target image based on the second detector of region of interest. In addition, the processing section 220 performs a process of outputting a third score indicating a probability that a region detected as the region of interest from the processing target image based on the observation method classifier. The processing section 220 then performs at least one of a process of integrating the first score and the third score and outputting a fourth score, or a process of integrating the second score and the third score and outputting a fifth score.

The first score mentioned herein is a detection score output from the first detector of region of interest. The second score is a detection score output from the second detector of region of interest. The third score is a detection score output from the detection integrated-type observation method classifier. The fourth score may be a higher score of the first score and the third score, a weighted sum, or another information obtained based on the first score and the third score. The fifth score may be a higher score of the second score and the third score, a weighted sum, or another information obtained based on the second score and the third score.

In a case where the first detector of region of interest is selected in the selection process, the processing section 220 then outputs a detection result based on the fourth score. In a case where the second detector of region of interest is selected in the selection process, the processing section 220 outputs a detection result based on the fifth score.

In this manner, the integration process in accordance with the present embodiment may be the integration process using scores. This enables appropriate and easy integration of an output from the detector of region of interest and an output from the detection integrated-type observation method classifier.

The observation method classifier is the trained model acquired by the machine learning based on the training image captured in the first observation method or the second observation method and the ground truth. The ground truth mentioned herein includes the detection data regarding at least one of whether the region of interest is present in the training image, and, if any, a position, a size, and a shape of the region of interest, and the observation method data indicating in which of the first observation method or the second observation method the training image is captured. In a case where there are three or more observation methods, the observation method classifier is the trained model acquired by the machine learning based on a training image captured in each of a plurality of observation methods and the ground truth. The observation method data is data indicating in which of the plurality of observation methods the trained model is captured.

This enables appropriate generation of the observation method classifier capable of outputting both the detection result and the result of observation method classification. As a result, the observation method classifier in accordance with the present embodiment is capable of executing the observation method classification process, and capable of executing the versatile detection process regardless of an observation method.

### 4. Third Embodiment

The above description has been given of the example of performing the processing targeting the two observation methods using the example of the normal light observation and the special light observation. However, there may be three or more observation methods in the present embodiment. In a third embodiment, a description will be given of an example in which observation methods include the normal light observation, the special light observation, and the pigment spray observation.

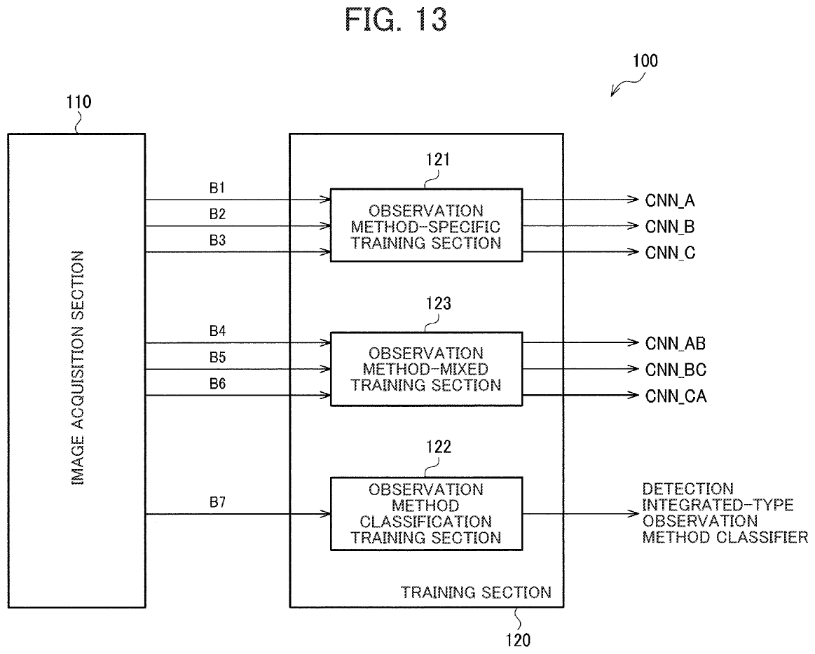

FIG. 13 illustrates a configuration example of the training device 100 in accordance with the third embodiment. The training section 120 of the training device 100 includes the observation method-specific training section 121, the observation method classification training section 122, and an observation method-mixed training section 123. Note that a configuration of the training device 100 is not limited to that illustrated in FIG. 13. Various modifications can be made such as omission of part of these constituent elements and addition of another constituent element. For example, the observation method-mixed training section 123 may be omitted.

A training process executed in the observation method-specific training section 121 is a training process for generating a trained model dedicated to any of observation methods. The observation method-specific training section 121 acquires an image group B1 from the image acquisition section 110, performs machine learning based on the image group B1, and thereby generates the first detector of region of interest. The observation method-specific training section 121 acquires an image group B2 from the image acquisition section 110, performs machine learning based on the image group B2, and thereby generates the second detector of region of interest. The observation method-specific training section 121 acquires an image group B3 from the image acquisition section 110, performs machine learning based on the image group B3, and thereby generates the third detector of region of interest.

The image group B1 is similar to the image group A1 illustrated in FIG. 7, and includes a training image in which the normal light image is provided with the detection data. The first detector of region of interest is a detector appropriate for the normal light image. The detector appropriate for the normal light image is hereinafter referred to as CNN_A.

The image group B2 is similar to the image group A2 illustrated in FIG. 7, and includes a training image in which the special light image is provided with the detection data. The second detector of region of interest is a detector appropriate for the special light image. The detector appropriate for the normal light image is hereinafter referred to as CNN_B.

The image group B3 includes a training image in which the pigment-sprayed image is provided with the detection data. The third detector of region of interest is a detector appropriate for the pigment-sprayed image. The detector appropriate for the pigment-sprayed image is hereinafter referred to as CNN_C.

The observation method classification training section 122 performs a training process for generating the detection integrated-type observation method classifier, similarly to, for example, the second embodiment. A configuration of the detection integrated-type observation method classifier is, for example, similar to that illustrated in FIG. 10. Note that since there are three or more observation methods in the present embodiment, the observation method classification layer outputs a result of the observation method classification indicating in which of the three or more observation methods the processing target image is captured.

An image group B7 is an image group including the training image in which the normal light image is provided with the detection data and the observation method data, a training image in which the special light image is provided with the detection data and the observation method data, and a training image in which the pigment-sprayed image is provided with the detection data and the observation method data. The observation method data is a label indicating which of the normal light image, the special light image, and the pigment-sprayed image the training image is.

The observation method-mixed training section 123 performs a training process for generating a detector of region of interest appropriate for two or more observation methods. Note that in the above-mentioned example, the detection integrated-type observation method classifier also serves as a detector of region of interest appropriate for all of the observation methods. Hence, the observation method-mixed training section 123 generates three detectors of region of interest comprising a detector of region of interest appropriate for the normal light image and the special light image, a detector of region of interest appropriate for the special light image and the pigment-sprayed image, and a detector of region of interest appropriate for the pigment-sprayed image and the normal light image. The detector of region of interest appropriate for the normal light image and the special light image is hereinafter referred to as CNN_AB. The detector of region of interest appropriate for the special light image and the pigment-sprayed image is hereinafter referred to as CNN_BC. The detector of region of interest appropriate for the pigment-sprayed image and the normal light image is hereinafter referred to as CNN_CA.

That is, an image group B4 illustrated in FIG. 13 includes a training image in which the normal light image is provided with the detection data, and a training image in which the special light image is provided with the detection data. The observation method-mixed training section 123 performs machine learning based on the image group B4 to generate the CNN_AB.

An image group B5 includes a training image in which the special light image is provided with the detection data, and a training image in which the pigment-sprayed image is provided with the detection data. The observation method-mixed training section 123 performs machine learning based on the image group B5 to generate the CNN_BC.

An image group B6 includes a training image in which the pigment-sprayed image is provided with the detection data, and a training image in which the normal light image is provided with the detection data. The observation method-mixed training section 123 performs machine learning based on the image group B6 to generate the CNN_CA.

A configuration of the image processing system 200 in accordance with the third embodiment is similar to that illustrated in FIG. 11. The image acquisition section 210 acquires an in-vivo image captured by the endoscope imaging device as a processing target image.

The detection classification section 225 performs calculation in the forward direction with the processing target image acquired by the image acquisition section 210 serving as the input to the detection integrated-type observation method classifier. The detection classification section 225 acquires information indicating a detection result from the detection layer and information indicating a result of the observation method classification from the observation method classification layer. The result of the observation method classification in accordance with the present embodiment is information that identifies which of the three or more observation methods the observation method for the processing target image is.

The selection section 222 selects a detector of region of interest based on the result of the observation method classification. In a case where the result of the observation method classification indicating that the processing target image is the normal light image is acquired, the selection section 222 selects the detector of region of interest using the normal light image as the training image. Specifically, the selection section 222 performs a process of selecting three detectors of region of interest comprising the CNN_A, the CNN_AB, and the CNN_CA. Similarly, in a case where the result of the observation method classification indicating that the processing target image is the special light image is acquired, the selection section 222 performs a process of selecting three detectors of region of interest comprising the CNN_B, the CNN_AB, and the CNN_BC. In a case where the result of the observation method classification indicating that the processing target image is the pigment-sprayed image is acquired, the selection section 222 performs a process of selecting three detectors of region of interest comprising the CNN_C, the CNN_BC, and the CNN_CA.

The detection processing section 223 performs a process of detecting the region of interest using the three detectors of region of interest selected by the selection section 222, and thereby acquires a detection result. That is, in the present embodiment, the detection processing section 223 outputs three patterns of detection results to the integration processing section 226.

The integration processing section 226 performs an integration process of integrating a detection result output from the detection classification section 225 using the detection integrated-type observation method classifier, and three detection results output from the detection processing section 223. Although the number of integration targets increases to four, the flow of the specific integration process is similar to that in the second embodiment. That is, the integration processing section 226 determines whether or not a plurality of detection frames correspond to an identical region of interest based on a degree of overlap of the detection frames. In a case of determining that the plurality of detection frames correspond to the identical region of interest, the integration processing section 226 performs a process of determining an integrated detection frame, and a process of determining a detection score to be associated with the detection frame.

As described above, the method in accordance with the present disclosure can be extended also to a case of using three or more observation methods. In this manner, integrating a plurality of detection results enables presentation of a detection result with higher accuracy.

In addition, the observation method in accordance with the present disclosure is not limited to the three types of observation comprising the normal light observation, the special light observation, and the pigment spray observation. For example, the observation method in accordance with the present embodiment may include water supply observation, air supply observation, bubble observation, residue observation, and the like. In the water supply observation method, imaging is performed in a state where a water supply operation to eject water from the insertion section. In the air supply observation method, imaging is performed in a state where an air supply operation to eject gas from the insertion section. In the bubble observation method, an image of a subject to which bubbles are attached is captured. In the residue observation method, an image of a subject to which a residue is attached is captured. A combination of observation methods can be flexibly changed, and two or more of the normal light observation, the special light observation, the pigment spray observation, the water supply observation, the air supply observation, the bubble observation, and the residue observation can be arbitrarily combined. Another observation method other than those described above may also be used.

### 5. Fourth Embodiment

For example, as a diagnosis step performed by a doctor, a step of searching for a lesion using the normal light observation and a step of differentiating a degree of malignancy of a detected lesion using the special light observation can be assumed. The special light image provides higher visibility of a lesion than that in the normal light image, and thus enables differentiation of the degree of malignancy with high accuracy. However, the number of special light images to be acquired is smaller than the number of the normal light images. For this reason, there is a possibility for a decrease in accuracy of detection due to insufficiency of training data in the machine learning using the special light image. For example, the accuracy of detection using the second detector of region of interest that has been trained with the special light image becomes lower than that using the first detector of region of interest that has been trained with the normal light image.

A method of performing pre-training and fine-tuning to supplement the insufficiency of the training data has been known. However, the conventional method does not take into consideration of a difference of observation methods between the special light image and the normal light image. Deep learning exhibits decreased recognition performance with respect to a test image captured under a condition that is different from that of an image group used for the training. The test image mentioned herein represents an image serving as a target of an inference process using a result of the training. That is, as the conventional method, a method of increasing accuracy of the detection process targeting the special light image has not been disclosed.

Hence, in the present embodiment, the pre-training using an image group including the normal light image is performed, thereafter the fine-tuning using an image group including the special light image is performed, and the second detector of region of interest is thereby generated. This can increase accuracy of detection even in a case where the special light image serves as a target of the detection process.

While the following description will be given of an example in which the first observation method is the normal light observation, and the second observation method is the special light observation, the second observation method may be the pigment spray observation. In addition, the second observation method can be extended to another observation method having a possibility for a decrease in detection accuracy due to insufficiency of training data. For example, the second observation method may be the air supply observation, the water supply observation, the bubble observation, the residue observation, or the like.

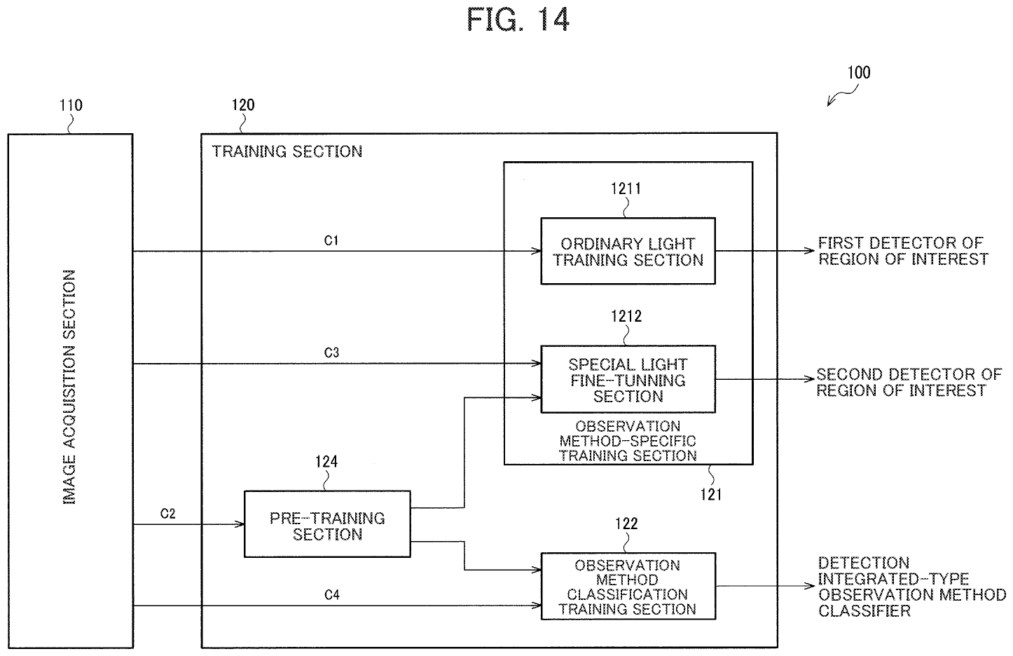

FIG. 14 illustrates a configuration example of the training device 100 in accordance with the present embodiment. The training section 120 includes the observation method-specific training section 121, the observation method classification training section 122, and a pre-training section 124. The observation method-specific training section 121 includes an normal light training section 1211 and a special light fine-tuning section 1212.

The normal light training section 1211 acquires an image group C1 from the image acquisition section 110, performs machine learning based on the image group C1, and thereby generates the first detector of region of interest. The image group C1 includes a training image in which the normal light image is provided with the detection data, similarly to the image groups A1 and B1. Training in the normal light training section 1211 is, for example, full-training that is not classified as the pre-training or the fine-tuning.

The pre-training section 124 performs pre-training using the image group C2. The image group C2 includes a training image in which the normal light image is provided with the detection data. As described above, the normal light observation is widely utilized in the step of searching for the region of interest. Thus, an abundance of normal light images provided with the detection data can be acquired. Note that the image group C2 may be an image group of training images that do not overlap with those of the image group C1, or may be an image group of training images, part or all of which overlap with those of the image group C1.

The special light fine-tuning section 1212 performs a training process using the special light images that are hard to be acquired in abundance. That is, the image group C3 is an image group including a plurality of training images in each of which the special light image is provided with the detection data. The special light fine-tuning section 1212 executes a training process using the image group C3 with a weight coefficient acquired by pre-training serving as an initial value, and thereby generates the second detector of region of interest appropriate for the special light image.

The pre-training section 124 may execute pre-training of the detection integrated-type observation method classifier. For example, the pre-training section 124 uses an image group including a training image in which the normal light image is provided with the detection data to perform pre-training for a detection task on the detection integrated-type observation method classifier. The pre-training for the detection task is a training process of using the detection data as the ground truth to update the weight coefficient of the feature amount extraction layer and the weight coefficient of the detection layer, which are illustrated in FIG. 10. That is, in the pre-training on the detection integrated-type observation method classifier, the weight coefficient of the observation method classification layer is not a target of training.

The observation method classification training section 122 executes fine-tuning using an image group C4 with the weight coefficient acquired by the pre-training serving as the initial value, and thereby generates the detection integrated-type observation method classifier. The image group C4 is an image group including a training image in which the normal light image is provided with the detection data and the observation method data and a training image in which the special light image is provided with the detection data and the observation method data, similarly to the second and third embodiments. That is, in the fine-tuning, the weight coefficients of all of the feature amount extraction layer, the detection layer, and the observation method classification layer serve as targets of training.

Processing after generation of the first detector of region of interest, the second detector of region of interest, and the detection integrated-type observation method classifier is similar to that in the second embodiment. Alternatively, the method in accordance with the fourth embodiment may be combined with the method in accordance with the third embodiment. That is, in a case where three or more observation methods including the normal light observation are used, pre-training using the normal light image and fine-tuning using a captured image in an observation method by which the number of images captured is insufficient can be combined.

As described above, the second detector of region of interest in accordance with the present embodiment is a trained model that has been trained by undergoing the pre-training using the first image group including an image captured in the first observation method and thereafter undergoing the fine-tuning using the second image group including an image captured in the second observation method. Note that the first observation method is preferably an observation method by which a large number of captured images are easily acquired, and is, specifically, the normal light observation. The second observation method is an observation method where training data tends to be insufficient, and may be the normal light observation as described above, the pigment spray observation, or another observation method.

In accordance with the method of the present embodiment, the pre-training of the machine learning is performed to supplement insufficiency of the number of training images. In a case where the neural network is used, the pre-training is a process of setting the initial value of the weight coefficient used when the fine-tuning is performed. This can increase accuracy of the detection process as compared with a case where the pre-training is not performed.

Alternatively, the observation method classifier may be a trained model that has been trained by undergoing the pre-training using the first image group including an image captured in the first observation method, and thereafter undergoing the fine-tuning using the third image group including an image captured in the first observation method and an image captured in the second observation method. In a case where there are three or more observation methods, the third image group includes a training image captured in each of a plurality of observation methods.

The first image group corresponds to the C2 in FIG. 14, and is, for example, an image group including a training image in which the normal light image is provided with the detection data. Note that an image group used for pre-training of the second detector of region of interest and an image group used for pre-training of the detection integrated-type observation method classifier may be different image groups. That is, the first image group may be an image group that is different from the image group C2 and that includes a training image in which the normal light image is provided with the detection data. The third image group corresponds to the C4 illustrated in FIG. 14, and is an image group including a training image in which the normal light image is provided with the detection data and the observation method data and a training image in which the special light image is provided with the detection data and the observation method data.

This can increase accuracy of the detection process in the detection integrated-type observation method classifier. The description has been given of the example of executing the pre-training and the fine-tuning in generation of both the second detector of region of interest and the detection integrated-type observation method classifier. However, the method in accordance with the present embodiment is not limited thereto. For example, one of the second detector of region of interest and the detection integrated-type observation method classifier may be generated by full-training. In a case where the present embodiment is combined with the third embodiment, a detector of region of interest other than the second detector of region of interest, for example, the CNN_AB, the CNN_BC, and the CNN_CA, may be generated using the pre-training and the fine-tuning.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An image processing system comprising a processor including hardware,
the processor being configured to
acquire a processing target image,
perform a classification process that classifies an observation method when the processing target image is captured to one of a plurality of observation methods including a first observation method and a second observation method based on an observation method classifier,
perform a selection process that selects one of a plurality of detectors of region of interest including a first detector of region of interest and a second detector of region of interest based on a classification result of the observation method classifier, each detector of region of interest detecting a region of interest from the processing target image,
output, when the first detector of region of interest is selected in the selection process, a detection result in which the region of interest is detected from the processing target image classified to the first observation method based on the first detector of region of interest, and
output, when the second detector of region of interest is selected in the selection process, the detection result in which the region of interest is detected from the processing target image classified to the second observation method based on the second detector of region of interest.

2. The image processing system as defined in claim 1, wherein the processor performs a process of detecting the region of interest from the processing target image based on the observation method classifier.

3. The image processing system as defined in claim 2,
wherein the processor
performs, when the first detector of region of interest is selected in the selection process, an integration process of the detection result of the region of interest based on the first detector of region of interest and the detection result of the region of interest based on the observation method classifier, and
performs, when the second detector of region of interest is selected in the selection process, the integration process of the detection result of the region of interest based on the second detector of region of interest and the detection result of the region of interest based on the observation method classifier.

4. The image processing system as defined in claim 3,
wherein the processor
performs at least one of a process of outputting a first score indicating a probability that a region detected as the region of interest from the processing target image is the region of interest based on the first detector of region of interest, or a process of outputting a second score indicating a probability that a region detected as the region of interest from the processing target image is the region of interest based on the second detector of region of interest,
performs a process of outputting a third score indicating a probability that a region detected as the region of interest from the processing target image is the region of interest based on the observation method classifier,
when the first detector of region of interest is selected in the selection process, integrates the first score and the third score to obtain a fourth score, and outputs the detection result based on the fourth score, and
when the second detector of region of interest is selected in the selection process, integrates the second score and the third score to obtain a fifth score, and outputs the detection result based on the fifth score.

5. The image processing system as defined in claim 1, wherein
the processing target image is an in-vivo image captured by an endoscope imaging device,
the first observation method is an observation method using normal light as illumination light, and
the second observation method is an observation method using special light as the illumination light.

6. The image processing system as defined in claim 1, wherein
the processing target image is an in-vivo image captured by an endoscope imaging device,
the first observation method is an observation method using normal light as illumination light, and
the second observation method is an observation method in which a pigment has been sprayed onto a subject.

7. The image processing system as defined in claim 1, wherein
the first detector of region of interest is a trained model acquired by machine learning performed based on a plurality of first training images captured in the first observation method, and detection data regarding at least one of whether the region of interest is present in each of the first training image, and, if any, a position, a size, and a shape of the region of interest in each of the first training image, and
the second detector of region of interest is a trained model acquired by machine learning performed based on a plurality of second training images captured in the second observation method, and the detection data in each of the second training image.

8. The image processing system as defined in claim 7, wherein the second detector of region of interest is the trained model having undergone pre-training using a first image group including an image captured in the first observation method, and thereafter having undergone fine-tuning using a second image group including an image captured in the second observation method.

9. The image processing system as defined in claim 3, wherein
the observation method classifier is a trained model acquired by machine learning based on a training image captured in either the first observation method or the second observation method and ground truth, and
the ground truth includes detection data regarding at least one of whether the region of interest is present in the training image, and, if any, a position, a size, and a shape of the region of interest, and observation method data indicating in which of the first observation method or the second observation method the training image is captured.

10. The image processing system as defined in claim 9, wherein the observation method classifier is the trained model that has been trained by undergoing pre-training using a first image group including an image captured in the first observation method, and thereafter undergoing fine-tuning using a third image group including an image captured in the first observation method and an image captured in the second observation method.

11. The image processing system as defined in claim 1, wherein at least one of the observation method classifier, the first detector of region of interest, or the second detector of region of interest comprises a convolutional neural network.

12. The image processing system as defined in claim 1, wherein the first detector of region of interest and the second detector of region of interest detect at least one of whether the region of interest is present, and, if any, a position of the region of interest, a size the region of interest, and a shape of the region of interest.

13. An endoscope system comprising:
an imaging device configured to capture an in-vivo image; and
a processor including hardware,
wherein the processor
acquires the in-vivo image as a processing target image,
performs a classification process that classifies an observation method when the processing target image is captured to one of a plurality of observation methods including a first observation method and a second observation method based on an observation method classifier,
performs a selection process that selects one of a plurality of detectors of region of interest including a first detector of region of interest and a second detector of region of interest based on a classification result of the observation method classifier, each detector of region of interest detecting a region of interest from the processing target image,
outputs, when the first detector of region of interest is selected in the selection process, a detection result in which the region of interest is detected from the processing target image classified to the first observation method based on the first detector of region of interest, and
outputs, when the second detector of region of interest is selected in the selection process, the detection result in which the region of interest is detected from the processing target image classified to the second observation method based on the second detector of region of interest.

14. An image processing method, comprising:
acquiring a processing target image,
performing a classification process that classifies an observation method when the processing target image is captured to one of a plurality of observation methods including a first observation method and a second observation method based on an observation method classifier,
performing a selection process that selects one of a plurality of detectors of region of interest including a first detector of region of interest and a second detector of region of interest based on a classification result of the observation method classifier, each detector of region of interest detecting a region of interest from the processing target image,
outputting, when the first detector of region of interest is selected in the selection process, a detection result in which the region of interest is detected from the processing target image classified to the first observation method based on the first detector of region of interest, and
outputting, when the second detector of region of interest is selected in the selection process, the detection result in which the region of interest is detected from the processing target image classified to the second observation method based on the second detector of region of interest.

15. A computer readable non-transitory storage medium that stores a program that causes a computer to execute steps comprising:
acquiring a processing target image,
performing a classification process that classifies an observation method when the processing target image is captured to one of a plurality of observation methods including a first observation method and a second observation method based on an observation method classifier,
performing a selection process that selects one of a plurality of detectors of region of interest including a first detector of region of interest and a second detector of region of interest based on a classification result of the observation method classifier, each detector of region of interest detecting a region of interest from the processing target image,
outputting, when the first detector of region of interest is selected in the selection process, a detection result in which the region of interest is detected from the processing target image classified to the first observation method based on the first detector of region of interest, and
outputting, when the second detector of region of interest is selected in the selection process, the detection result in which the region of interest is detected from the processing target image classified to the second observation method based on the second detector of region of interest.
